(19) United States
(12) Patent Application Publication
(10) Pub. No.: US 2024/0065255 A1
(43) Pub. Date: Feb. 29, 2024

(54) CONTROLLING A CROP CARE IMPLEMENT OF A WORK VEHICLE

(71) Applicant: Deere & Company, Moline, IL (US)

(72) Inventor: Noel W. Anderson, Fargo, ND (US)

(21) Appl. No.: 17/823,581

(22) Filed: Aug. 31, 2022

(51) Int. Cl. A01M 7/00; G01N 33/00; G01S 19/01; G06V 20/10

(52) U.S. Cl. CPC A01M 7/0089; G01N 33/0098; G01S 19/01; G06V 20/188

(57) ABSTRACT

A work vehicle configured for operating in a field for growing a crop. The work vehicle comprises an implement coupled to the work vehicle. A global positioning system is communicatively coupled to the work vehicle. The global positioning system is configured for generating a location signal indicative of a location of the work vehicle. A sensor is communicatively coupled to the work vehicle. The sensor is configured for sensing a current field characteristic and generating a current field characteristic signal indicative of the field characteristic. A control system is communicatively coupled to the work vehicle. The control system is configured to receive the location signal, receive the current field characteristic signal, determine a sentinel plant characteristic of a sentinel plant from the current field characteristic, determine an implement action based on the sentinel plant characteristic, and control the implement to execute the implement action in the field.

### FIELD OF THE DISCLOSURE

The present disclosure generally relates to controlling a crop care implement of a work vehicle and more particularly to controlling a ground engaging implement of the work vehicle using sentinel plants in a field.

### BACKGROUND OF THE DISCLOSURE

Work vehicles with ground engaging implements include crop care machines such as a sprayer that is generally used to deliver a substance to a field using a plurality of nozzles. Sprayers may be self-propelled or pulled by another work vehicle.

### SUMMARY OF THE DISCLOSURE

In one embodiment, a work vehicle configured for operating in a field for growing a crop is disclosed. The work vehicle comprises an implement coupled to the work vehicle. A global positioning system is communicatively coupled to the work vehicle. The global positioning system is configured for generating a location signal indicative of a location of the work vehicle. A sensor is communicatively coupled to the work vehicle. The sensor is configured for sensing a current field characteristic and generating a current field characteristic signal indicative of the current field characteristic. A control system is communicatively coupled to the work vehicle. The control system is configured to receive the location signal, receive the current field characteristic signal, determine a sentinel plant characteristic of a sentinel plant from the current field characteristic, determine an implement action based on the sentinel plant characteristic, and control the implement to execute the implement action in the field.

In another embodiment, a work vehicle configured for operating in a field for growing a crop is disclosed. The work vehicle comprises an implement coupled to the work vehicle. A global positioning system is communicatively coupled to the work vehicle. The global positioning system is configured for generating a location signal indicative of a location of the work vehicle. A sensor is communicatively coupled to the work vehicle. The sensor is configured for sensing a sentinel plant characteristic and generating a sentinel plant characteristic signal indicative of the sentinel plant characteristic. A control system is communicatively coupled to the work vehicle. The control system is configured to receive the location signal, receive the sentinel plant characteristic signal, determine an implement action based on the sentinel plant characteristic, and control the implement to execute the implement action in the field.

In yet another embodiment, a method of controlling a work vehicle is disclosed. The method comprises sensing a georeferenced first sentinel plant characteristic of a first sentinel plant. Determining an implement action based on the first sentinel plant characteristic and controlling the implement to execute the implement action.

Other features and aspects will become apparent by consideration of the detailed description and accompanying drawings.

### BRIEF DESCRIPTION OF THE DRAWINGS

FIG. 1 is a side view of a self-propelled sprayer;

FIG. 2 is a rear view of the self-propelled sprayer of FIG. 1;

FIG. 3 is a block diagram of a sprayer; and

FIG. 4 is a flow diagram of a method for operating a work vehicle.

Before any embodiments are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Further embodiments of the invention may include any combination of features from one or more dependent claims, and such features may be incorporated, collectively or separately, into any independent claim.

As used herein, unless otherwise limited or modified, lists with elements that are separated by conjunctive terms (e.g., “and”) and that are also preceded by the phrase “at least one of” or “one or more of” indicate configurations or arrangements that potentially include individual elements of the list, or any combination thereof. For example, “at least one of A, B, and/or C” or “one or more of A, B, and/or C” indicates the possibilities of only A, only B, only C, or any combination of two or more of A, B, and C (e.g., A and B; B and C; A and C; or A, B, and C).

### DETAILED DESCRIPTION

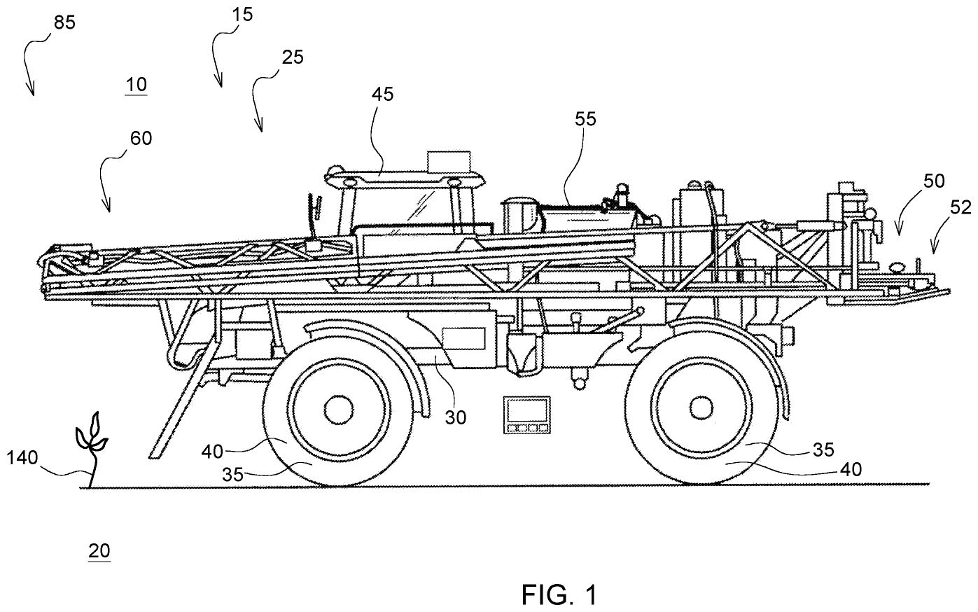

FIG. 1 illustrates a work vehicle 10. The illustrated work vehicle 10 is a crop care machine in the form of a sprayer 15 configured for spraying crops on a field or surface 20. Other types of work vehicles 10, including other types of crop care machines, are contemplated by this disclosure including construction equipment (e.g., wheel loader, crawler), road building equipment (e.g., motor grader), forestry equipment (e.g., tracked feller buncher) and agricultural equipment (e.g., chisel plow).

The illustrated sprayer 15 is a self-propelled sprayer 25. Other types of sprayers 15 are contemplated by this disclosure (e.g., pull behind sprayer, dry spreader). The self-propelled sprayer 25 comprises a frame 30. The frame 30 is supported by at least one ground-engaging element 35. The ground-engaging element 35 may be a wheel 40 or a track (not shown). An operator station 45 is also coupleable to the frame 30.

An implement 50 is coupled to the frame 30. The illustrated implement 50 may be a spray assembly 52 that comprises a tank 55 that comprises a volume for storing at least one substance (e.g., chemical) to be dispensed on the surface 20. Alternatively, the tank 55 may comprise a plurality of separate volumes for providing a plurality of potentially different substances. For example, one substance may be a herbicide while another substance might be a fungicide or pesticide. The one or more substances can be selected to manage attributes of the soil or crop, such as soil fertility, soil quality, water, weeds, pests, diseases, biodiversity, wildlife, and other attributes.

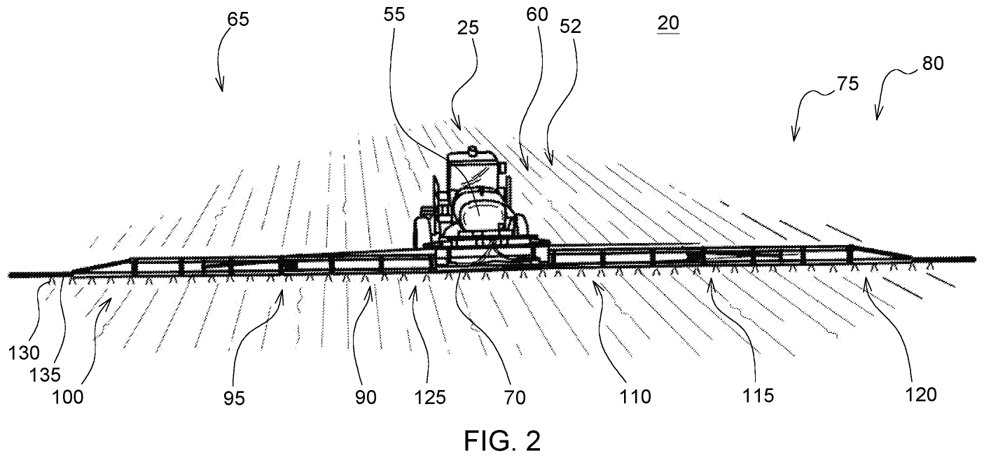

The spray assembly 52 further comprises a boom assembly 60 that is in fluid communication with the tank 55. With reference to FIG. 2, the boom assembly 60 comprises a left boom 65, a center frame 70, and a right boom 75. Other boom assembly 60 configurations are contemplated by this disclosure. The boom assembly 60 is configured to unfold into a working configuration 80 for spraying the substance onto the surface 20 and fold into a transport configuration 85 (FIG. 1) when not spraying.

In the illustrated configuration, the left boom 65 comprises a left inner boom 90 that is coupled to the center frame 70. A left center boom 95 is coupled to the left inner boom 90. A left outer boom 100 is coupled to the left center boom 95. Other left boom 65 configurations are contemplated by this disclosure. In the working configuration 80, the left inner boom 90 is placed into an end-to-end relationship in a longitudinal direction 105 with the left center boom 95 while the left outer boom 100 is also placed into an end-to-end relationship in the longitudinal direction 105 with the left center boom 95. In the transport configuration 85, the left outer boom 100 is folded into a facing relationship, and is parallel with, the left center boom 95. The left center boom 95 is folded into a facing relationship, and is parallel with, the left inner boom 90.

In the illustrated configuration, the right boom 75 comprises a right inner boom 110 that is coupled to the center frame 70. A right center boom 115 is coupled to the right inner boom 110. A right outer boom 120 is coupled to the right center boom 115. Other right boom 75 configurations are contemplated by this disclosure. In the working configuration 80, the right inner boom 110 is placed into an end-to-end relationship in the longitudinal direction 105 with the right center boom 115 while the right outer boom 120 is also placed into an end-to-end relationship in the longitudinal direction 105 with the right center boom 115. In the transport configuration 85, the right outer boom 120 is folded into a facing relationship, and is parallel with, the right center boom 115. The right center boom 115 is folded into a facing relationship, and is parallel with, the right inner boom 110.

A nozzle assembly 125 is coupled to the left boom 65, the center frame 70, and the right boom 75. The nozzle assembly 125 comprises a plurality of nozzles 130 and a plurality of conduits 135 for delivering the substance from the tank 55 to the plurality of nozzles 130. The nozzle assembly 125 is configured to deliver the substance to a plant 140 (FIG. 1) one at a time or to a plurality of plants 140 at one time. Other types of applicators besides nozzles 130 are contemplated by this disclosure (e.g., drop applicators).

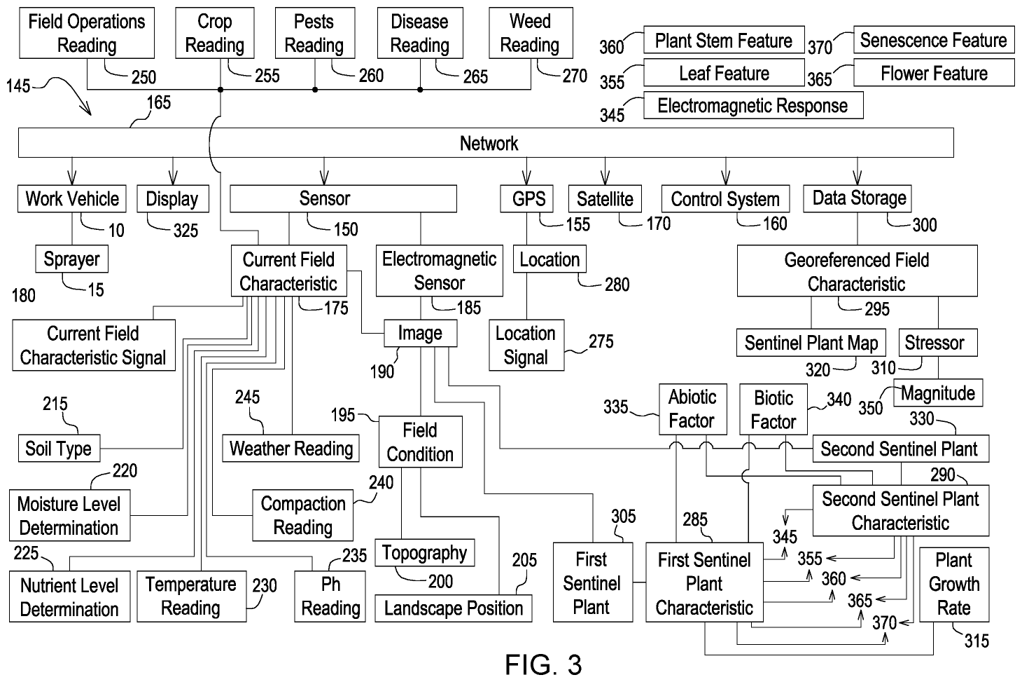

Referring to FIG. 3, a block diagram is provided of one example of a computing architecture 145 that includes the sprayer 15, a sensor 150, a global positioning system (“GPS”) 155, and a control system 160. Global positioning system 155 may comprise a Global Navigation Satellite System (GNSS), a terrestrial radio triangulation system, or any other system which is able to provide the location of the sprayer 15 in the field 20 in global or local coordinates. FIG. 3 illustratively shows that the sprayer 15, the sensor 150, the GPS 155, and the control system 160 are connected over a network 165. Thus, computing architecture 145 operates in a networked environment, where the network 165 includes any of a wide variety of different logical connections such as a local area network (LAN), wide area network (WAN), controller area network (CAN) near field communication network, satellite communication network, cellular networks, or a wide variety of other networks or combination of networks. It is also noted that the control system 160 can be deployed on the sprayer 15 such that the control system 160 performs the operations described herein without a networked connection. In addition, while the present description will primarily focus on an example of the control system 160 communicating with the sprayer 15, it is noted that the same or similar functionality can be provided when communicating with a wide variety of work vehicles 10 and/or remote systems. Work vehicles 10 may comprise without limitation, planters, seeders, chemical applicators, sprayer, rotary hoes, plows, disks, harvesters, soil samplers, scouting robots, or unmanned aerial vehicles.

The sensor 150 may be communicatively coupled to the work vehicle 10. For example, the sensor 150 may be coupled to the sprayer 15 or it may be coupled to a satellite 170 that communicates with the work vehicle 10 or control system 160. In some examples, the satellite 170 may comprise a manned aircraft or unmanned aerial vehicle as a remote sensor platform. The sensor 150 may be configured for sensing a current field characteristic 175 and generating a current field characteristic signal 180 indicative of the current field characteristic 175. The sensor 150 may include an electromagnetic sensor 185 and the current field characteristic 175 may include an image 190 that depicts a field condition 195. The image 190 may be 2D or 3D. The image 190 may comprise a single pixel or data value. The image 190 may comprise a sentinel plant characteristic such as the first sentinel plant characteristic or the second sentinel plant characteristic 290. The electromagnetic sensor 185 may be a camera or other imaging sensor such as a video camera. Alternatively, the sensor 150 may include at least one of a radar, lidar, laser-based sensors, LIDAR based sensors, temperature sensors, soil property sensors, NIR sensor, visible light sensor, UV sensor, and a wide variety of other imaging or other sensing systems.

The current field characteristic 175 may include the image 190 that depicts the field condition 195 such as a topography 200 of the surface 20 or a landscape position 205 of plants 140 or a size of the plant 140 patches. The current field characteristic 175 may also include soil type 215 such as clay composition or top soil composition. The current field characteristic 175 may include a moisture level determination 220 such as 20-80% water content or a nutrient level determination 225 that includes a level of soil organic matter, or residue cover, or cover crops. Additionally, the current field characteristic 175 may include a temperature reading 230 of the surface 20 at a soil depth or a pH reading 235 of the soil of the surface 20.

The current field characteristic 175 may include a compaction reading 240 of the surface 20 that includes compaction layer details such as depth of the compaction and compaction drainage and its location in the surface 20. Additionally, the current field characteristic 175 may include a weather reading 245. The weather reading 245 may include past, present, and future temperatures, moisture, solar, sun angle, sunlight attenuation by clouds or other, day length, or other.

The current field characteristic 175 may include a field operations reading 250 that indicates machinery data including machinery settings or timing of machinery operations along with the location in the surface 20. Additionally, the current field characteristic 175 may include a crop reading 255 that indicates crop details including mono or multiple varieties that are planted in the surface 20 and their location. The current field characteristic 175 may include a pests reading 260 including an indication of any insects, mammals, birds, or other similar biotic factors and their location in the surface 20. The current field characteristic 175 may include a disease reading 265 including an indication of any bacteria, molds, smuts, viruses or other similar biotic factors and their location in the field. The current field characteristic 175 may include a weed reading 270 including an indication of any weeds and weed seeds and their location in the surface 20. Other current field characteristics 175 are contemplated by this disclosure.

The GPS 155 is communicatively coupled to the sprayer 15. The GPS 155 is configured for generating a location signal 275 indicative of a location 280 of the sprayer 15 or work vehicle 10. Generally, the GPS 155 receives sensor signals from one or more sensors, such as a GPS receiver, a dead reckoning system, a LORAN system, or a wide variety of other systems or sensors, to determine the location 280 of the sprayer 15 across the surface 20.

The control system 160 is in communication with the sensor 150 and the GPS 155. The control system 160 is communicatively coupled to the sprayer 15. The control system 160 may be configured to receive a first sentinel plant characteristic 285, receive a second sentinel plant characteristic 290, receive the location signal 275, the current field characteristic 175, receive a georeferenced field characteristic 295 from a data storage 300, and control at least one of the plurality of nozzles 130 based on at least one of the first sentinel plant characteristic 285, the second sentinel plant characteristic 290, the location 280, the current field characteristic 175, or the georeferenced field characteristic 295, or any combination thereof. In other embodiments, the control system 160 may control other implement 50 actions such as a substance application location or rate. The georeferenced field characteristic may include a depth and spacing information for the plants 140 based on how they were planted in a previous operation or based on historical data for the field or surface 20. The georeferenced field characteristic 295 may include relationships between first and second sentinel plant characteristics 285, 290 that can be sensed and the georeferenced field characteristics 295 that promote a degree or a magnitude 350 of response to a stressor 310.

The control system 160 may be configured to control the plurality of nozzles 130 to spray at or near a first sentinel plant 305 at the location where the georeferenced field characteristic 295 indicates the stressor 310 for the first sentinel plant characteristic 285. For example, the georeferenced field characteristic 295 may indicate an area or location of the surface 20 where there is or may be a high incidence of an insect. The control system 160 may control the plurality of nozzles 130 to spray at or near the first sentinel plant 305 that has the first sentinel plant characteristic 285 that is elicited by the insect at this location. The first sentinel plant characteristic 285 may be a slow plant growth rate 315.

In an additional example, the georeferenced field characteristic 295 may indicate an area of the surface 20 where there is a high probability of soybean chlorosis in some years. The control system 160 may control the plurality of nozzles 130 to spray at or near the first sentinel plant 305 that has the first sentinel plant characteristic 285 that is elicited by difficulties in plant uptake of iron, resulting in chlorosis, at this location. The first sentinel plant characteristic 285 may be a yellowing of leaves proportional to the severity of the stress. Mitigation could be to apply chelated iron and maybe sulfur with the sprayer 15, the work vehicle 10, or other equipment.

The georeferenced field characteristic 295 may include a sentinel plant map 320 and the control system 160 may be configured to update the sentinel plant map 320 and the georeferenced field characteristic 295 in the data storage 300 with the current field characteristic 175. In some examples, sentinel plant map 320 comprises locations where sentinel plants were planted in the past. This data may be used to correctly identify and interpret images 190 containing first and second sentinel plant characteristics 285, 290. The sensor 150 may be used to obtain the data stored in the data storage 300. The data storage 300 may be coupled to the sprayer 15, the work vehicle 10, or the satellite 170, or may be located at some other location. In some examples, satellite 170 may comprise a manned aircraft or unmanned aerial vehicle as a remote sensor platform.

A display 325 may be provided for displaying the sentinel plant map 320 to an operator. Additionally, the operator may enter sprayer 15 or work vehicle 10 commands via the display 325. Display 325 may include audio elements such as a speaker or microphone. Display 325 may include haptic elements such as a vibration generator.

A planter (not shown) may be used to plant the first sentinel plant 305 or seed, a second sentinel plant 330 or seed, and other seeds in proximity of, or near, the first sentinel plant 305 and/or the second sentinel plant 330. The first and second sentinel plants 305, 330 exhibit different characteristics when presented with the stressor 310. The different first sentinel plant characteristic 285 and second sentinel plant characteristic 290 can be any detectable plant attribute that is proportional to the stressor 310 such as an abiotic factor 335 or a biotic factor 340. For example, the abiotic factor 335 may be a lack of or an abundance of water, humidity, light, nutrients, or minerals. The abiotic factor 335 may also be soil conditions or temperature. The biotic factor 340 may be the presence of a certain fungus, bacteria, or insect.

For example, the first and second sentinel plants 305, 330 may be genetically modified corn or soy beans that exhibit different characteristics from the other corn or soy beans that the first and second sentinel plants 305, 330 are planted with, respectively. In one example of genetic modification, sentinel plants may be modified to produce green fluorescent protein (“GFP”) in response to a stressor. In other examples, the seed may naturally have or be traditionally bred to have a differentiated characteristic response to a stressor 305. The planter may be configured for planting the first sentinel plant 305 that exhibits the first sentinel plant characteristic 285. The first sentinel plant 305 may be a sentinel seed (not shown) or a sentinel seedling (not shown) or any other sentinel plant form. A sentinel plant may also be a fungus, a lichen, mold, or any other stationary life form. The planter may also be configured for planting the second sentinel plant 305 that exhibits the second sentinel plant characteristic 290. The second sentinel plant characteristic 290 may be the same as the first sentinel plant characteristic 285 or it may vary. The second sentinel plant 330 may be a sentinel seed (not shown) or a sentinel seedling (not shown) or any other sentinel plant form. A sentinel plant may also be a fungus, a lichen, mold, or any other stationary life form.

In one embodiment, the first or second sentinel plant characteristic 285, 290 may include at least one of varying intensity of electromagnetic response 345 generated by the first or second sentinel plant 305, 330 or the plant growth rate 315 that correspond to a magnitude 350 of the stressor 310. The electromagnetic response 345 may be an absorption, transmission, backscatter, reflectance, fluorescence, bioluminescence, or other. The electromagnetic response 345 may be induced with or without external stimulation.

In another embodiment, the first or second sentinel plant characteristic 285, 290 may include a change in a leaf feature 355 such as a change in leaf size, color, color pattern, shape, area index, or temperature. Other leaf features 355 are contemplated by this disclosure.

In yet another embodiment, the first or second sentinel plant characteristic 285, 290 may include a change in a plant stem feature 360 such as a change in plant stem height, plant stem biomass, plant stem formations or malformations, plant stem diameter, plant stem color, or plant stem color pattern. Other plant stem features 365 are contemplated by this disclosure.

The first or second sentinel plant characteristic 285, 290 may include the slow plant growth rate 315 or otherwise altered growth rate, or a time to emergence, seed germination rate, or an initiation of reproductive phase.

In another embodiment, the first or second sentinel plant characteristic 285, 290 may include a change in a flower feature 365 such as flower color or flower bloom timing. Other flower features 365 are contemplated by this disclosure.

In yet another embodiment, the first or second sentinel plant characteristic 285, 290 may include a senescence feature 370 such as death of the sentinel plant due to daylight, a daylight change derivative, a change in temperature, a soil chemical attribute, or other. Other first or second sentinel plant characteristics 285, 290 are contemplated by this disclosure.

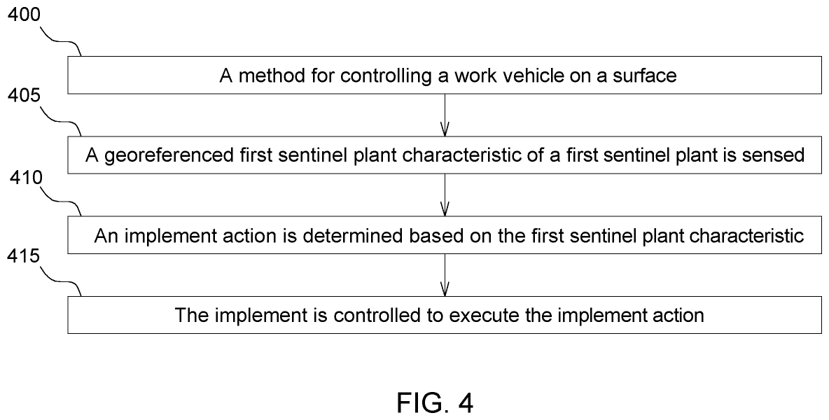

Referring now to FIG. 4, a flow diagram of a method 400 for controlling a work vehicle 10 on a surface 20 is provided. At 405, a georeferenced first sentinel plant characteristic 295 of a first sentinel plant 305 is sensed. At 410, an implement 50 action is determined based on the first sentinel plant characteristic 295. At 415, the implement 50 is controlled to execute the implement 50 action.

The first sentinel plant characteristic 285 may comprise an image 190 of the first sentinel plant 305 that includes at least one of the stressor 310 or the magnitude 350 of the stressor 310 of the first sentinel plant 305. The stressor 310 may comprise at least one of an abiotic factor 335 or a biotic factor 340. The first sentinel plant characteristic 285 may comprise at least one of varying intensities of an electromagnetic response 345 generated by the first sentinel plant 305 or a plant growth rate 315 that corresponds to the magnitude 350 of the stressor 310. The electromagnetic response 345 may be an absorption, transmission, backscatter, reflectance, fluorescence, bioluminescence, or other. The implement 50 may comprise at least one of a plurality of spray nozzles 130 and the implement action may comprise spraying a substance through at least one of a plurality of spray nozzles 130. The substance may be a liquid or a solid. Other implement actions are contemplated by this disclosure.

Advantageously, the first or second sentinel plants 305, 330 would not need to be planted at the same time as the main crop. They could be planted at, say, the time of spring tillage ahead of the crop planting pass. They could also be planted after the main crop. In one example, a problem is otherwise identified in the surface 20 and then the first or second sentinel plants 305, 330 are planted to play a diagnostic role to find out why the stand is poor or lagging. In another example, the first or second sentinel plants 305, 330 could be planted just prior to a critical crop phase such as pollination to provide data on conditions during a time window. The first or second sentinel plants 305, 330 could also be perennials planted once for multiple seasons of use for annual crops or multi-year crops like cane sugar.

The first or second sentinel plants 305, 330 could be an organism, other than plants, such as bacteria or fungi (some soil fungi in the tropics are naturally fluorescent). The response of the sentinel organisms could be detected by sensors on a ground engaging element such as a shank. Similarly, the response to the stressor 310 by the first or second sentinel plants 305, 330 may be in the roots and observed below ground.

The first or second sentinel plants 305, 330 could depend on allelopathy. A first plant, micro, fungi could be the one which is proportionally responsive to the stressor 310. The response may be in the form of proportionally altering the environment around a second plant. The second plant then communicates the level of that alternate environment. For example, in response to a soil condition, a microbe alters the pH of the soil adjacent to it. This causes a color change in the flower of a co-located plant sensitive to soil pH. A liquid may be precisely applied in the seed trench and planting the first or second sentinel plants 305, 330 may comprise placing a seed and inoculating the soil near the seed with bacteria, fungi, etc. The inoculant wouldn't even need to be alive. It could be the first plant is replaced by a chemical that reacts with one or more soil components, living or non-living, that results in a proportional amount that is reported via the second plant. Chemical may be applied as a liquid, solid, or gas. Also note that the reporter plant response can be other things besides fluorescence such as a nitrogen level experienced by a microbe, a local soil pH level, or a plant flower or leaf color.

Machine learning could be used to improve the efficacy of where to plant plants 210 including the first or second sentinel plants 305, 330. The ability of the sentinel plants to accurately sense desired crop conditions, in some cases compared to laboratory analysis or other sensing means, may be improved with machine learning. This improvement could be in the selection of varieties to use as sentinels, planting attributes (e.g., interplant spacing, depth), or location of individual plants 210 or plant 210 patches.

In one example, sprayer 15 is moving across surface 20. Electromagnetic sensor 185 captures an image 190, which includes plant 140. Plant 140 is georeferenced with location 280 from GPS 155 by control system 160. Control system 160 accesses sentinel plant map 320 and determines that plant 140 is first sentinel plant 305. Control system 160 analyzes image 190 using sentinel plant information in data storage 300. Crop reading 255, shade and coverage of yellow in leaves, is consistent with a stressor 310 of magnitude 350 for iron chlorosis. Control system 160 then retrieves corresponding remediation prescription from data storage 300 and commands sprayer 15 to apply an amount of chelated iron to crop in vicinity of plant 140.

What is claimed is:

1. A work vehicle configured for operating in a field for growing a crop, the work vehicle comprising:
an implement coupled to the work vehicle;
a global positioning system communicatively coupled to the work vehicle, the global positioning system configured for generating a location signal indicative of a location of the work vehicle;
a sensor communicatively coupled to the work vehicle, the sensor configured for sensing a current field characteristic and generating a current field characteristic signal indicative of the current field characteristic; and
a control system communicatively coupled to the work vehicle, the control system configured to:
receive the location signal,
receive the current field characteristic signal,
determine a sentinel plant characteristic of a sentinel plant from the current field characteristic,
determine an implement action based on the sentinel plant characteristic, and
control the implement to execute the implement action in the field.

2. The work vehicle of claim 1, wherein the sensor comprises an electromagnetic sensor, the current field characteristic comprises an image of the crop, and the sentinel plant characteristic comprises an image of the sentinel plant included in a portion of the image of the crop that includes at least one of a stressor or a magnitude of the stressor of the sentinel plant.

3. The work vehicle of claim 2, wherein the stressor comprises at least one of an abiotic factor or a biotic factor.

4. The work vehicle of claim 2, wherein the sentinel plant characteristic comprises at least one of varying intensities of electromagnetic response generated by the sentinel plant or a plant growth rate that correspond to the magnitude of the stressor.

5. The work vehicle of claim 1, wherein the implement comprises at least one spray nozzle and the implement action comprises spraying a substance through at least one spray nozzle.

6. The work vehicle of claim 1, wherein the sensor comprises an electromagnetic sensor configured for taking an image of a field condition.

7. The work vehicle of claim 1, wherein the control system records the location to a data storage, records the sentinel plant characteristic to the data storage, and generates a sentinel plant map.

8. A work vehicle configured for operating in a field for growing a crop, the work vehicle comprising:
an implement coupled to the work vehicle;
a global positioning system communicatively coupled to the work vehicle, the global positioning system configured for generating a location signal indicative of a location of the work vehicle;
a sensor communicatively coupled to the work vehicle, the sensor configured for sensing a sentinel plant characteristic and generating a sentinel plant characteristic signal indicative of the sentinel plant characteristic; and
a control system communicatively coupled to the work vehicle, the control system configured to:
receive the location signal,
receive the sentinel plant characteristic signal,
determine an implement action based on the sentinel plant characteristic, and
control the implement to execute the implement action in the field.

9. The work vehicle of claim 8, wherein the sensor comprises an electromagnetic sensor, the sentinel plant characteristic comprises an image of the sentinel plant that includes at least one of a stressor or a magnitude of the stressor of the sentinel plant.

10. The work vehicle of claim 9, wherein the stressor comprises at least one of an abiotic factor or a biotic factor.

11. The work vehicle of claim 9, wherein the sentinel plant characteristic comprises at least one of varying intensities of electromagnetic response generated by the sentinel plant or a plant growth rate that correspond to the magnitude of the stressor.

12. The work vehicle of claim 8, wherein the implement comprises at least one spray nozzle and the implement action comprises spraying a substance through at least one spray nozzle.

13. The work vehicle of claim 12, wherein the substance is a liquid.

14. The work vehicle of claim 8, wherein the control system records the location to a data storage, records the sentinel plant characteristic to the data storage, and generates a sentinel plant map.

15. The work vehicle of claim 8, wherein the implement comprises at least one of a plurality of spray nozzles and the implement action comprises spraying a substance through at least one of a plurality of spray nozzles.

16. A method of controlling a work vehicle operating on a surface, the method comprising:
sensing a georeferenced first sentinel plant characteristic of a first sentinel plant;
determining an implement action based on the first sentinel plant characteristic; and
controlling the implement to execute the implement action.

17. The method of claim 16, wherein the first sentinel plant characteristic comprises an image of the first sentinel plant that includes at least one of a stressor or a magnitude of the stressor of the first sentinel plant.

18. The method of claim 17, wherein the stressor comprises at least one of an abiotic factor or a biotic factor.

19. The method of claim 17, wherein the first sentinel plant characteristic comprises at least one of varying intensities of electromagnetic response generated by the first sentinel plant or a plant growth rate that corresponds to the magnitude of the stressor.

20. The method of claim 16, wherein the implement comprises at least one of a plurality of spray nozzles and the implement action comprises spraying a substance through at least one of a plurality of spray nozzles.
